(19) United States
(12) Patent Application Publication
(10) Pub. No.: US 2007/0208050 A1
(43) Pub. Date: Sep. 6, 2007

(54) PROCESS FOR PREPARING IRINOTECAN

(76) Inventors: Venkata Raghavendra Acharyulu Palle, Hyderabad (IN); Sekhar Munaswamy Nariyam, Hyderabad (IN); Lankeshwararao Matti, Hyderabad (IN); Raghupati Rama Subrahmanyam Vinjamuri, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/678,071

(22) Filed: Feb. 23, 2007

Related U.S. Application Data
(60) Provisional application No. 60/805,255, filed on Jun. 20, 2006.

(30) Foreign Application Priority Data
Feb. 24, 2006 (IN) 310/CHE/2006

(51) Int. Cl. A61K 31/4745; C07D 491/14

(52) U.S. Cl. 514/283; 546/52

(57) ABSTRACT

The present invention relates to a process for the preparation of pure irinotecan or salts thereof, and a process for the preparation of intermediate compound 7-ethyl-10-hydroxycamptothecin.

### INTRODUCTION TO THE INVENTION

The present invention relates to processes for the preparation of irinotecan and its salts. In an embodiment, it relates to processes for the preparation of irinotecan hydrochloride trihydrate substantially free of impurities.

Irinotecan has the chemical name (S)-4,11-diethyl-3,4,12,14-tetrahydro-4-hydroxy-3,14-dioxo1H-pyrano[3′,4′:6,7]-indolizino-[1,2-b]-quinolin-9-yl-[1,4′-bipiperidine]-1′-carboxylate, (hereafter referred to by the adopted name “irinotecan”) and has the structure represented in Formula I.

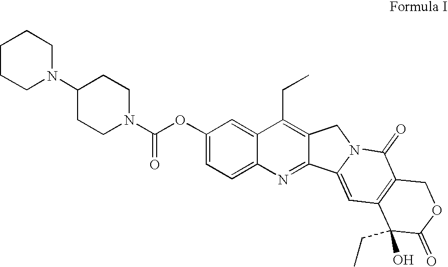

Irinotecan is a camptothecin derivative that was proven to be highly active against lung and colorectal cancer and has been used for treating several types of tumors. It is sold in the form of the hydrochloride salt trihydrate under the trade name CAMPTOSAR® and is available in the form of vials containing 40 mg or 100 mg of irinotecan hydrochloride, in the form of aqueous solutions.

U.S. Pat. No. 4,604,463 discloses irinotecan and its pharmaceutical acceptable salts. The patent also describes a process for the preparation of irinotecan, which comprises the condensation of 7-ethyl-10-hydroxy camptothecin with 1-chlorocarbonyl-4-piperidinopiperidine in presence of pyridine at room temperature.

U.S. Pat. No. 5,633,260 discloses 7-ethyl 10-hydroxy camptothecin an intermediate compound of Formula II used in the preparation of irinotecan hydrochloride trihydrate and a process for its preparation. It describes the process for the preparation of 7-ethyl 10-hydroxy camptothecin by using ferrous sulphate heptahydrate, propanal and hydrogen peroxide from 10-hydroxy camptothecin, the product being purified by expensive, laborious column chromatography procedures.

Satoru Okajima et al., Chemical Pharmaceutical Bulletin, Japan, Vol. 39 (6), pages 1446-1454, 1991 describes the preparation of irinotecan hydrochloride trihydrate.

None of the above mentioned processes disclose an industrial process for the purification of irinotecan and its intermediates to make them substantially free from structure related impurities.

Thus there is a long felt need to develop a process for the preparation of irinotecan substantially free from structure related impurities.

The present invention provides convenient processes for purification of irinotecan and its intermediates with desired purity and yield, which are economical and industrially feasible.

### SUMMARY OF THE INVENTION

The present invention relates to processes for the preparation of irinotecan and its salts substantially free from impurities.

In one aspect the present invention provides a process for preparing irinotecan, comprising:

i) slurrying 7-ethyl-10-hydroxycamptothecin in alcohol;

ii) dissolving 7-ethyl-10-hydroxycamptothecin in acetic acid, removing acetic acid to form a concentrated solution, and adding alcohol to form a precipitate;

iii) recrystallizing 7-ethyl-10-hydroxycamptothecin of Formula II; and

iv) reacting 7-ethyl-10-hydroxycamptothecin with 4-piperidinopiperidine carbamoyl chloride to form irinotecan.

In another aspect the present invention provides a process for purifying irinotecan, comprising adding a solution comprising irinotecan to a column of silica gel, eluting using an eluent comprising dichloromethane, then eluting using an eluent comprising a mixture of methanol and dichloromethane.

### BRIEF DESCRIPTION OF THE DRAWINGS

FIG. 1 is an X-ray powder diffraction pattern of irinotecan hydrochloride trihydrate prepared according to Example 11.

FIG. 2 is a differential scanning calorimetry curve of irinotecan hydrochloride trihydrate prepared according to Example 11.

FIG. 3 is an infrared absorption spectrum of irinotecan hydrochloride trihydrate prepared according to Example 11.

FIG. 4 is a thermogravimetric analysis curve of irinotecan hydrochloride trihydrate prepared according to Example 11.

### DETAILED DESCRIPTION OF THE INVENTION

The present invention relates to a process for the preparation of irinotecan and its salts substantially free from impurities.

In one aspect the present invention provides a process for preparing irinotecan, comprising:

i) slurrying 7-ethyl-10-hydroxycamptothecin of Formula II in an alcohol; then

ii) dissolving 7-ethyl-10-hydroxycamptothecin of Formula II in acetic acid, removing acetic acid to form a concentrated solution, and adding alcohol to form a precipitate; then

iii) recrystallizing 7-ethyl-10-hydroxycamptothecin of Formula II; and

iv) reacting 7-ethyl-10-hydroxycamptothecin with 4-piperidinopiperidine carbamoyl chloride to form irinotecan.

Step i) involves slurrying 7-ethyl-10-hydroxycamptothecin of Formula II in an alcohol.

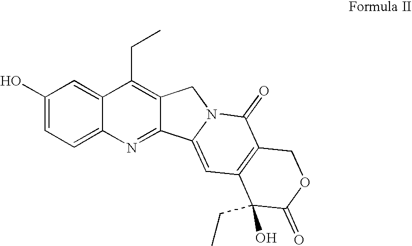

The slurry is a suspension comprising the compound 7-ethyl-10-hydroxycamptothecin and an alcohol. The slurrying can be carried out for any desired time periods to achieve a desired purity, such as from about 20 minutes to about 5 hours, or longer.

Alcohols, which can be used to form a slurry, include without limitation thereto C₁-C₆ alkyl alcohols such as methanol, ethanol, isopropanol, n-butanol and the like.

Suitably, the slurrying is carried out for the purification of 7-ethyl-10-hydroxy camptothecin at temperatures from about 20 to about 50° C., or about 20 to about 35° C.

7-ethyl-10-hydroxy camptothecin of Formula II obtained by the above process of step i) of present invention has a purity of not less than about 90%, or about 92%, by high performance liquid chromatography (“HPLC”).

Step ii) involves dissolving 7-ethyl-10-hydroxycamptothecin of Formula II in acetic acid, removing acetic acid to form a concentrated solution, and combining with an alcohol to form a precipitate.

Concentrations of acetic acid used for dissolution of 7-ethyl-10-hydroxycamptothecin are from about 95 to about 100%.

The concentrations of 7-ethyl-10-hydroxycamptothecin in the acetic acid is not critical as long as sufficient acetic acid is employed to ensure total dissolution. The amount of acetic acid employed is ordinarily kept as low as possible to avoid excessive product loss during crystallization and isolation. The quantity of acetic acid used for the dissolution of 7-ethyl-10-hydroxycamptothecin frequently is about 3 to about 32 times the weight of 7-ethyl-10-hydroxycamptothecin.

The solution can be prepared at any temperatures up to the boiling point of the solvent. Frequently, the solution will be prepared at an elevated temperature for enhanced solute concentration.

Removal of the acetic acid may be carried out suitably using evaporation, atmospheric distillation, or distillation under vacuum.

Suitably, the removal of acetic acid is carried out at temperatures from about 35 to about 100° C., or about 45 to about 55° C., for times sufficient to achieve a desired concentration.

The removal of acetic acid is carried out until the solute concentration reaches about 0.1 g/ml to about 0.3 g/ml

The acetic acid solution can be combined with an alcohol to cause precipitation of solid.

Alcohols, which can be used in formation of precipitate, include without limitation thereto C₁-C₆ alkyl alcohols such as methanol, ethanol, isopropanol, n-butanol and the like.

The quantity of alcohol that can be used to isolate the solid can range from about 1 to about 5 volumes, per volume of the acetic acid solution.

7-ethyl-10-hydroxy camptothecin of Formula II obtained by the above process of step ii) of the present invention has a purity of not less than about 93%, or about 95%, by HPLC.

Step iii) involves recrystallizing 7-ethyl-10-hydroxycamptothecin of Formula II.

Solvents, which can be used in the crystallization of 7-ethyl-10-hydroxycamptothecin, include but are not limited to N,N-dimethylformamide (DMF), dimethylsulfoxide (DMSO), and N,N-dimethylacetamide (DMA).

The quantity of solvent used for dissolution depends on the nature of solvent and the temperature adopted for preparing the solution. The concentration of 7-ethyl-10-hydroxy camptothecin in the solution may generally range from about 0.01 to about 10 g/ml.

Dissolution of 7-ethyl-10-hydroxycamptothecin can be carried out in a temperature range from about 30 to 120° C., or about 70 to 80° C.

For crystallization to occur, the solution may be maintained further at temperatures lower than the dissolution temperatures such as, for example, below about 10° C. to about 35° C., for a period of time as required for a more complete isolation of the product. The exact cooling temperature and time required for complete isolation can be readily determined by a person skilled in the art and will also depend on parameters such as concentration and temperature of the solution or slurry.

Optionally, isolation may be enhanced by methods such as cooling, partial removal of the solvent from the mixture, seeding, adding an anti-solvent to the reaction mixture, or any combination thereof.

The solid can be recovered using any techniques such as filtration by gravity or by suction, centrifugation, decantation, and the like.

The obtained wet cake optionally can be slurried in a suitable solvent to remove non-volatile components.

Suitable solvents that can be used in providing a slurry include without limitation thereto C₁-C₆ alkyl alcohols such as methanol, ethanol, isopropanol, 1-propanol, n-butanol and the like.

7-ethyl-10-hydroxycamptothecin of Formula II obtained by the process of step iii) of the present invention has a purity of not less than about 95%, or about 98%, by high performance liquid chromatography (“HPLC”).

Step iv) involves reaction of the compound 7-ethyl-10-hydroxycamptothecin with 4-piperidinopiperidine carbamoylchloride to form irinotecan.

The formation of irinotecan can be carried out in the presence of a base, such as pyridine, triethylamine, diethylamine and the like.

Suitably, the reaction can be carried out at temperatures of about 20 to about 60° C., or about 25 to about 45° C.

After completion of the formation of irinotecan, the reaction mixture is optionally subjected to pH adjustment with a acid to extract the reaction mixture into an aqueous medium and a solid can be isolated by combining with an alcohol. Useful alcohol antisolvents include methanol, ethanol, isopropyl alcohol, n-butanol and the like.

The pH of the reaction mixture for conducting the reaction can range from about 3 to about 7, or from about 5 to about 6.

Acids, which can be used for the extraction of reaction mixture into an aqueous layer, include but are not limited to acetic acid, trifluoroacetic acid, hydrochloric acid, hydrobromic acid, sulfuric acid and the like.

Irinotecan of Formula I obtained by the above process of the present invention has a purity of not less than about 90%, or about 94%, by HPLC.

In another aspect, the present invention provides process for purifying irinotecan, comprising adding a solution comprising irinotecan and suitable solvent to a column of silica gel, eluting using an eluent comprising dichloromethane, then eluting using a eluent comprising a mixture of methanol and dichloromethane.

The silica gel, which can be used for the purposes of this invention, can have a particle size range such as for example 230-400 mesh, 100-200 mesh, 60-100 mesh, or 500-750 mesh. The quantity of silica gel used can range from about 4 to 20 times, or about 4 to 6 times, the weight of the irinotecan.

The silica gels used in the adsorption steps can be identical or different. Selection of silica gel depends upon the properties of the silica gel like pore size, grain size, surface area, polarity of the surface, type of material to be purified, level and nature of impurities present, type of medium, the type of mobile phase.

Resins can also be used for adsorption instead of silica gel. Suitable resins which can be used for the purpose of the present invention include commercially available resins like those manufactured by Rohm and Haas, Philadelphia, Pa. U.S.A., such as AMBERLITE™ XAD 4, XAD 7, XAD 16, XAD 1 6HP, XAD 761, and XAD 1180. Other resins suitable for the process of the invention include those manufactured and sold by Mitsubishi Kasei Corporation, Japan, such as DAION™ HP 10, DAION HP 20, DAION HP 21, DAION HP 30, DAION HP 40, DAION HP 50, DAION SP 800, DAION SP 825, DAION SP 850, DAION SP 875, DAION SP 205, DAION SP 207, DAION HP1MG, and DAION HP2MG.

The resins which can be used also include divinylbenzene-styrene copolymers or copolymers of divinylbenzene, styrene and other derivatives of these having aliphatic and/or aromatic moieties comprising from 2 to 18 carbon atoms, or having substituted halogen atoms such as chlorine, fluorine or bromine, or, a copolymer of divinylbenzene and styrene with surface grafted moieties that are aliphatic or aromatic containing 2 or more carbon atoms and/or having substituted halogen atoms such as chlorine, fluorine or bromine, resins based on one or combination of natural polymers, derivatized or as such from for example, agarose, dextran or cellulose, resins based on polymethacrylate matrix, or combinations with other acrylate polymers, prepared by cross-linking of monomers, with or without grafted moieties that are aliphatic or aromatic containing 2 or more carbon atoms with or without substituted halogen atoms from chlorine, fluorine or bromine.

The adsorbent silica gel may be filled into any suitable container or reaction vessel such as for example a filtration funnel or a cylindrical vessel or the like. The size of the container may be chosen based on the amount of the silica gel to be contained therein and the batch size of irinotecan to be purified.

Stabilizing the column is achieved by passing a suitable solvent through the column at a desired flow rate. Suitable solvents for elution include, without limitation thereto: chlorinated solvents such as dichloromethane, dichloroethane, chloroform, and the like; alcohols such as isopropanol, methanol, and ethanol; hydrocarbons such as cyclohexane; and the like; esters such as ethyl acetate and n-butyl acetate; ketones such as acetone and the like; ethers such as 1,4-dioxane, dimethoxyethane, tetrahydrofuran and the like; hydrocarbons such as n-hexane, n-heptane and the like; and mixtures thereof. Mixtures of such solvents, in various proportions, are useful. The temperature of the solvent used for elution can range from about 5 to 80° C. The appropriate temperature to be used will be determined based on the solvent or solvent mixture that is used to achieve optimal separation. The flow of the solvent can be continuous or in lots and the quantity of solvent can range from about 2 to 20 times the volume of the solution containing the crude irinotecan.

Column efficiency is determined by calculating the theoretical plates comprising injecting pure irinotecan solution or injecting the mixture of suitable phthalate components. Suitable phthalates include dibutyl phthalate, diethyl phthalate, dimethyl phthalate and the like.

The solution of crude irinotecan can be prepared by the dissolving irinotecan comprising impurities in a suitable solvent or in suitable solvent mixture. Examples of useful solvents include methanol, ethanol, isopropanol, n-butanol, dichloromethane, chloroform and mixtures thereof.

Crude irinotecan is adsorbed onto the silica gel by passing the solution through a silica gel bed or column. The silica gel loaded with crude irinotecan is then eluted using a suitable solvent or a mixture of solvents and the eluted solvent fraction containing the purified irinotecan is collected.

Suitable solvents which can be used in the elution include but are not limited to methanol, ethanol, isopropanol, n-butanol, dichloromethane, chloroform and mixtures thereof. In an embodiment, elution is conducted first with dichloromethane, then with a mixture of about 5 percent by volume methanol in dichloromethane.

The solid can be recovered from pure fractions by conventional techniques such evaporation, atmospheric distillation or distillation under vacuum, and solid can also be isolated by adding anti solvent such as n-hexane, cyclohexane, n-heptane and the like.

Irinotecan of Formula I obtained by the process of present invention has a purity of not less than about 98%, or about 99%, by HPLC.

Irinotecan can be further purified using a recrystallization technique with a suitable solvent, which process comprises:

a) providing a solution of irinotecan;

b) increasing the concentration of solution of step a) to cause precipitation; and

c) recovering the irinotecan of Formula I.

The step a) involves providing a solution of irinotecan.

The step of providing a solution of crude irinotecan includes dissolving solid irinotecan in a suitable solvent or obtaining a solution comprising crude irinotecan from a previous processing step such as from preparative column chromatography.

The concentration of irinotecan in the solvent is not critical as long as sufficient solvent is employed to ensure total dissolution. The amount of solvent employed is usually kept to a minimum so as to avoid excessive product loss during crystallization and isolation.

Suitable solvents which can be used for providing a solution of irinotecan include but are not limited to: chlorinated solvents such as dichloromethane, dichloroethane, chloroform, and the like; alcohols such as methanol, ethanol, isopropyl alcohol, n-butanol and the like; and mixtures thereof.

The solution can be prepared at temperatures ranging from about 25° C. to 100° C. Depending on the quantity of solvent taken, irinotecan may dissolve at 25 to 100° C., or the solution may need to be heated to elevated temperatures of about 50° C. to 100° C.

A decolorizing carbon treatment can be optionally given either at the dissolution temperatures or after cooling the solution to lower temperatures.

Step b) involves increasing the concentration of the solution of step a) to cause precipitation;

Concentration may be carried out suitably using evaporation, atmospheric distillation or distillation under vacuum.

Distillation of the solvent may be conducted under a vacuum of about 100 mm Hg to about 600 mm Hg at temperatures of about 40° C. to about 70° C. Any temperature and vacuum conditions can be used as long as concentration occurs without increasing the impurity levels.

Concentration of the solution can be carried out to an extent where the precipitation of the irinotecan begins from the solution, converting the solution into slurry. Generally, concentration will be terminated when solution concentration reaches to about 0.1 g/ml to about 0.3 g/ml.

The mixture may be maintained further at temperatures lower than the concentration temperatures such as for example below about 40° C. to about 45° C., for a period of time as required for a more complete isolation of the product. The exact cooling temperature and time required for complete crystallization can be readily determined by a person skilled in the art and will also depend on parameters such as concentration and temperature of the solution or slurry.

Step c) involves recovering the irinotecan of Formula I.

The solid can be recovered by conventional techniques such as filtering, decanting, centrifuging and the like, or by filtering under an inert atmosphere using gases such as for example nitrogen and the like.

The above recrystallization steps can be repeated two or more times to get a purity greater than or equal to about 99.5%, and the irinotecan can then be further converted into the pharmaceutical acceptable salts using suitable acids such as hydrochloric acid, hydrobromic acid, acetic acid, trifluoroacetic acid, and the like.

In one embodiment, the present invention provides a process for preparation of irinotecan hydrochloride trihydrate by reacting irinotecan with hydrochloric acid.

Hydrochloric acid that can be used includes aqueous solutions or alcoholic solutions. Alcoholic solutions can be prepared by using alcohols such as methanol, ethanol, isopropyl alcohol, n-butanol and the like.

Irinotecan or its hydrochloride salt or irinotecan hydrochloride trihydrate obtained by the above process has been analyzed using high performance liquid chromatography (“HPLC”) with the conditions described in Table 1.

TABLE 1

| Column and | Waters symmetry shield C18, 250 × 4.6 mm, 5 | | |
|---|---|---|---|
| Packing: | microns | | |
| Buffer | 2.72 g of potassium dihydrogen phosphate in 1000 ml | | |
| | of water and pH adjusted to 3.5 ± 0.05 with dilute | | |
| | phosphoric acid. | | |
| Mobile Phase A: | Used buffer as mobile phase A | | |
| Mobile Phase B: | Mixture of acetonitrile and methanol in the ratio of | | |
| | 60:40 (% v/v). | | |
| Gradient: | Time | Solution A | Solution B |
| | (minutes) | (% v/v) | (% v/v) |
| | 0 | 80 | 20 |
| | 40 | 30 | 70 |
| | 45 | 30 | 70 |
| | 50 | 80 | 20 |

| | |
|---|---|
| Flow rate: | 1.0 ml/minute |
| Wavelength of | 220 nm by UV |
| detection: | |
| Temperature: | 25 ± 2° C. |
| Injection volume: | 10 μL |
| Sample | 50 mg of sample is dissolved in diluent and diluted to |
| preparation | 50 ml. |
| Diluent: | Mixed buffer, acetonitrile and methanol in the ratio of |
| | 50:25:25 (v/v) |
| Run time: | 50 minutes |
| Post time: | 5 minutes |

The following relative retention time (“RRT”) values are obtained for irinotecan impurities, when the retention time for each impurity peak is divided by the retention time for irinotecan (RRT=1):

| Impurity | RRT |
|---|---|
| Camptothecin | 1.38 |
| 10-hydroxy camptothecin | 1.19 |
| 7-ethyl10-hydroxy camptothecin | 1.53 |
| 7-ethyl camptothecin | 1.8 |
| 7,11-diethyl10-hydroxy | 2.1 |
| camptothecin | |
| 10-[4-(1-piperidino)-piperidino]- | 0.8 |
| carbonyloxy camptothecin | |
| 7,11-diethyl-10-[4(1-piperidino)-1- | 1.26 |
| piperidino]- | |
| carbonyloxycamptothecin | |

The process according to the invention provides substantially pure irinotecan with a purity greater than or equal to about 99.5% by HPLC.

Irinotecan or its hydrochloride salt, provided by the process of this invention has a purity by high performance liquid chromatography (“HPLC”) greater than or equal to about 99.5%. It contains less than about 0.15%, or less than about 0.0015%, of (S)-4-ethyl-4-hydroxy-1H-pyrano[3′,4′:6,7] indolizino(1,2-b)quinoline-3,14(4H,12H)-dione (“camptothecin”) of Formula III;

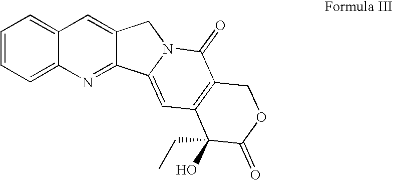

less than about 0.15%, or less than about 0.0015%, of (S)-4-ethyl-4,9-dihydroxy-1H-pyrano[3′,4′:6,7]indolizino(1,2-b)quinoline-3,14(4H,12H)-dione, or 10-hydroxy camptothecin, of Formula IV;

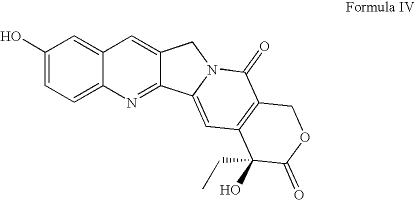

less than about 0.15%, or less than about 0.0015%, of (S)-4,11-diethyl-4,9-dihydroxy-1H-pyrano[3′,4′:6,7]indolizino(1,2-b)quinoline-3,14(4H,12H)-dione, or 7-ethyl-10-hydroxy camptothecin, of Formula II;

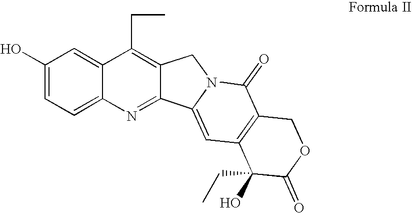

less than about 0.15%, or less than about 0.005%, of (S)-4,11-diethyl-4-hydroxy-1H-pyrano[3′,4′:6,7]indolizino(1,2-b)quinoline-3,14(4H,12H)-dione, or 7-ethyl camptothecin, of Formula V;

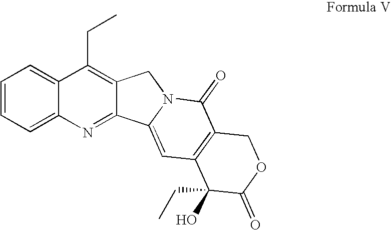

less than about 0.15%, or less than about 0.0025%, of (S)-4,8,11-triethyl-4,9-dihydroxy-1H-pyrano[3′,4′:6,7]indolizino(1,2-b)quinoline-3,14(4H,12H)-dione, or 7,11-diethyl-10-hydroxy camptothecin, of Formula VI;

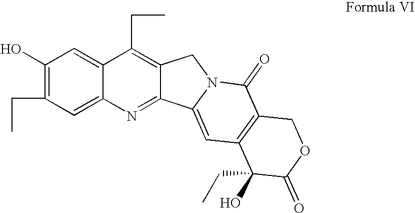

less than about 0.15%, or less than about 0.003%, of (S)-4-ethyl-4,9-dihydroxy-1H-pyrano[3′,4′:6,7]indolizino(1,2-b)quinoline-3,14(4H,12H)-dione-9-(1,4′-bipiperidine)-1′-carboxylate, or 10-[4-(1-piperidino)-piperidino]-carbonyloxy camptothecin, of Formula VIII; and

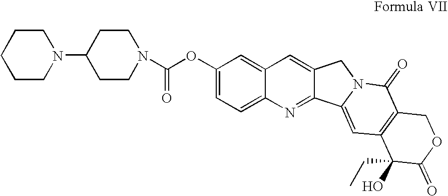

less than about 0.15%, or less than about 0.005%, of (S)-4,8,11-triethyl-4,9-dihydroxy-1H-pyrano[3′,4′:6,7]indolizino(1,2-b)quinoline-3,14(4H,12H)-dione-9-(1,4′-bipiperidine)-1′-carboxylate, or 7,11-diethyl-10-[4(1-piperidino)-1-piperidino]-carbonyloxycamptothecin, of Formula VIII.

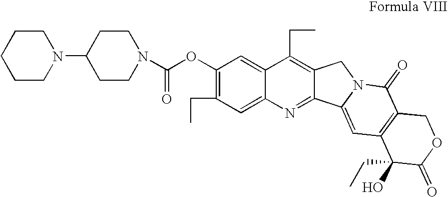

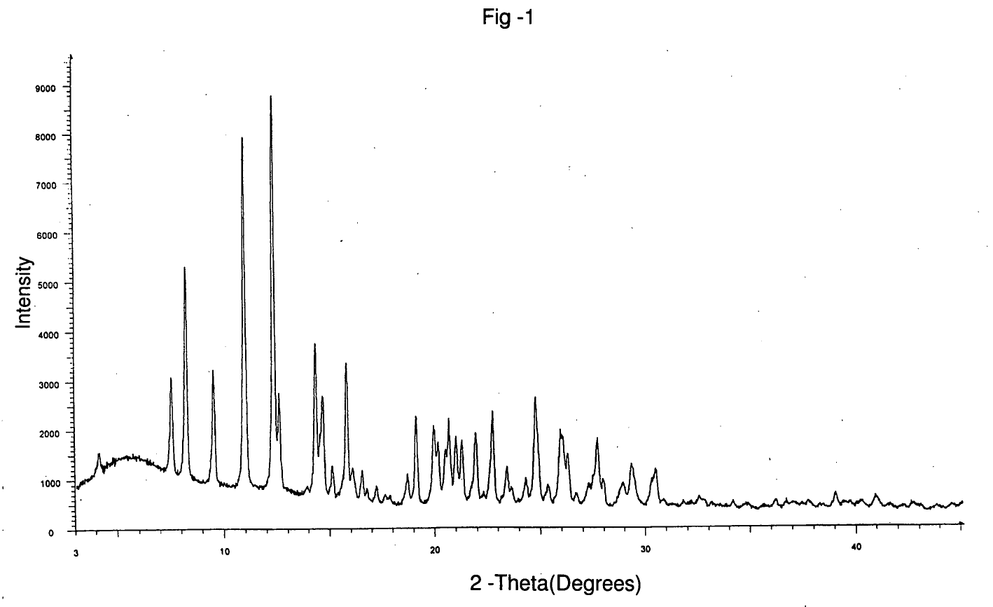

Crystalline irinotecan hydrochloride trihydrate obtained from the process of the present invention is characterized by its XRPD pattern, substantially in accordance with the pattern of FIG. 1. All XRPD data reported herein were obtained using Cu Kα radiation, having the wavelength 1.541 Å and were obtained using a Bruker AXS D8 Advance Powder X-ray Diffractometer.

Crystalline irinotecan hydrochloride trihydrate is characterized by an XRPD diffraction pattern comprising characteristic peaks at about 8.2, 9.5, 11.0, 12.4, 14.4, 14.7, 15.8, 22.7, and 24.8, ±0.2 degrees two-theta.

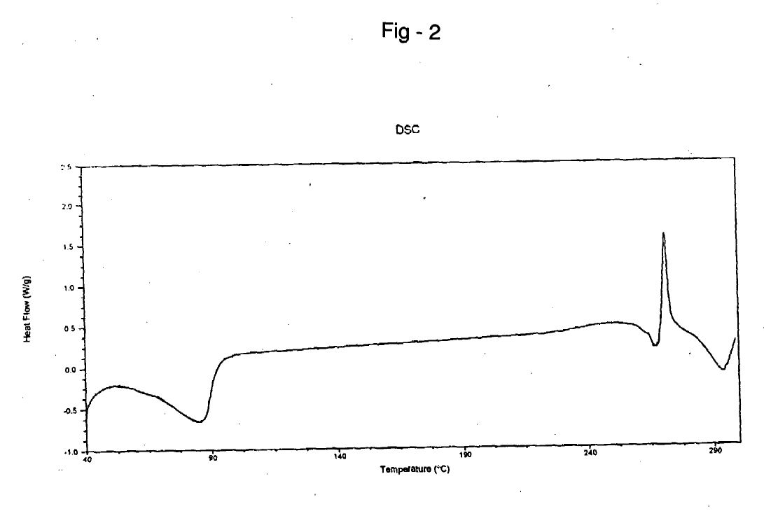

Crystalline irinotecan hydrochloride trihydrate of the present invention has a characteristic differential scanning calorimetric curve substantially in accordance with the curve of FIG. 2. Crystalline irinotecan hydrochloride trihydrate of the present invention has a characteristic differential scanning calorimetric curve having endothermic peaks at about 86° C. and 267° C. (with decomposition).

Differential scanning calorimetric analysis was carried out in a DSC Q1000 Model from TA Instruments with a ramp of 5° C./minute with a modulation time of 60 seconds and a modulation temperature of ±1° C. The starting temperature was 0° C. and ending temperature was 200° C.

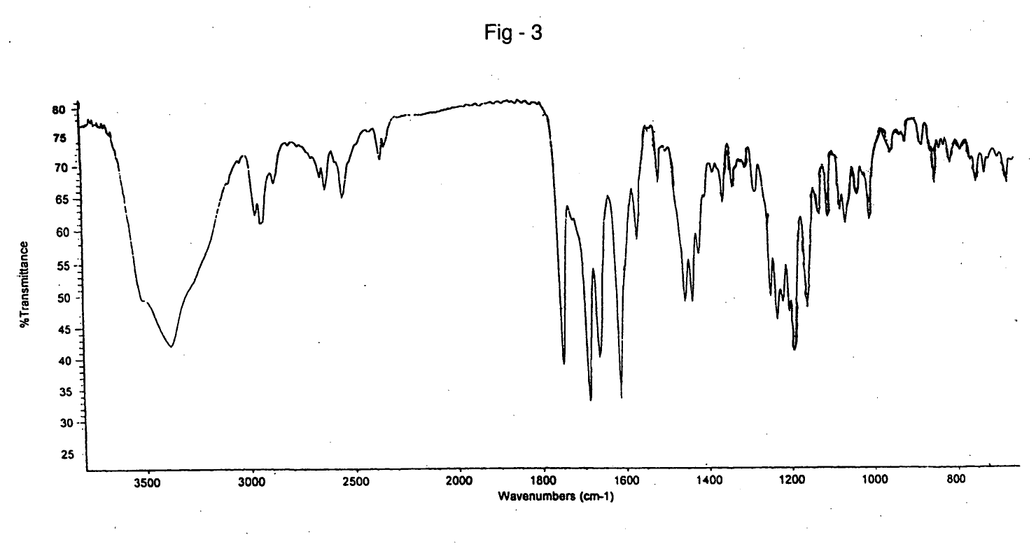

In yet a further aspect, the present invention provides irinotecan hydrochloride trihydrate with a high purity having an infrared absorption spectrum in a potassium bromide (KBr) pellet as represented by the spectrum of FIG. 3.

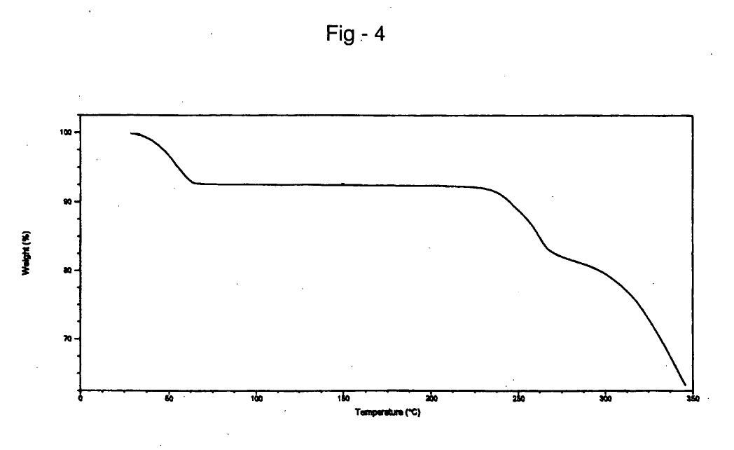

Crystalline irinotecan hydrochloride trihydrate of the present invention has a characteristic thermogravimetric analysis curve (TGA) corresponding to a weight loss of about 7.5% w/w, substantially in accordance with FIG. 4, showing that it is in a trihydrate form.

In an embodiment, irinotecan hydrochloride trihydrate obtained by the process of the present invention has an average particle length less than about 50 microns, or about 30 microns, or about 10 microns.

A method for determining particle length involves attaching 5-10 mg of sample to one side of a double-side adhesive tape, attaching the tape to aluminum foil, coating with gold and viewing the particles using a scanning electron microscope.

Irinotecan hydrochloride trihydrate obtained by the process of the present invention contains amounts of residual solvents that are within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

Irinotecan hydrochloride obtained in this invention contains: less than about 100 ppm, or less than about 500 ppm, of methanol; less than about 100 ppm, or less than about 500 ppm, of isopropyl alcohol; less than about 100 ppm, or less than about 500 ppm, of ethanol; less than about 100 ppm, or less than about 500 ppm, of n-heptane; less than about 100 ppm, or less than about 500 ppm, of dichloromethane; less than about 100 ppm, or less than about 500 ppm, of ethyl acetate; less than about 50 ppm, or less than about 100 ppm, of pyridine; and less than about 50 ppm, or less than about 100 ppm, of N,N-dimethylformamide.

The process for preparing pure irinotecan hydrochloride of the present invention is simple, efficient, industrially feasible, eco-friendly and provides a final compound having reproducible high purity.

Certain specific aspects and embodiments of the invention will be explained in more detail with reference to the following examples, which are provided by way of illustration only and should not be construed as limiting the scope of the invention in any manner.

### EXAMPLE 1

### PREPARATION OF 10-HYDROXY CAMPTOTHECIN

6 L of acetic acid and 60 ml of thioanisole were taken into a reactor and 2 Kg of camptothecin suspended in 6 L of acetic acid was charged. 666 g of platinum oxide was suspended in 6 L of acetic acid in another container and then the suspension was charged to the reactor at 27.5° C. The container was washed with 2 L of acetic acid and then charged into the reactor. The obtained reaction mixture was subjected to evacuation and then a pressure of 45 psi was applied using hydrogen gas, which was then slowly increased to 65 psi at 34.3° C. and the mixture was stirred for 10 minutes. The reaction mixture was heated to 61.9° C. and stirred for 6 hours at 63.7° C. and at 64 psi. Then the reaction mass was allowed to cool to 41.3° C. with stirring for 22 hours at 62 psi. Reaction completion was confirmed by thin layer chromatography (“TLC”). After completion of the reaction, the hydrogen pressure was slowly released and the reactor was flushed with nitrogen gas. Then the reaction mixture was filtered through a flux calcined diatomaceous earth (“Hyflow”) bed followed by washing with 6.6 L of acetic acid.

26.6 L of water was added to the filtrate containing tetrahydrocamptothecin of Formula III. 4 Kg of iodosobenzene diacetate was added to the solution and subjected to stirring for about 18 hours at 27.5° C. The reaction mixture was distilled at 115° C. until 13 L remained (distillate quantity 41.5 L). The reaction mixture was allowed to cool to 27.9° C. Then the reaction suspension was filtered and the wet solid washed with 4 L of methanol.

The resultant wet solid and 12 L of dimethylformamide were charged into a flask and the suspension was subjected to heating to 76.3° C. The suspension was stirred for 20 minutes for complete dissolution at 76.3° C. and the obtained solution was stirred for 1 hour at 76.5° C. The solution was allowed to cool to 27.5° C. and 59.5 L of methanol was charged and then subjected to stirring for about 5 hours at 27.5° C. The mass was filtered and the solid washed with methanol, followed by suction drying. The wet solid material was transferred into a flask and 6 L of methanol was charged, followed by raising the temperature to 62.5° C. and maintaining at these conditions for about 1 hour. The mass was cooled to 27.5° C. and then the solid was filtered. The flask was rinsed with 3 L of methanol and then the wet solid was washed with the above rinsing. The obtained solid was suction dried and dried at 57.8° C. under high vacuum at 680 mm Hg for 12 hours to afford 1070 g of the title compound having a purity of about 97.4% by HPLC.

### EXAMPLE 2

### PREPARATION OF 7-ETHYL-10-HYDROXY CAMPTOTHECIN (FORMULA II)

125 g of 10-hydroxy camptothecin and 37.5 g of ferrous sulfate heptahydrate were charged into 3.75 L of water. 78.5 ml of propanal was charged into above solution with simultaneous stirring for about 10 minutes. The resultant reaction mixture was cooled to about 0° C. 1500 ml of concentrated sulfuric acid was added dropwise to the reaction mass over a period of 4 hours followed by the addition of 200 ml of 30% hydrogen peroxide solution at 0° C. over a period of 45 minutes. The reaction mass was allowed to reach 27° C. with simultaneous stirring for about 2 hours and then cooled to about 0° C. After the completion of the reaction, 7.2 L of 18% aqueous sodium hydroxide solution was added at 20° C. with simultaneous stirring over a period of 45 minutes. The solid separated was filtered and the solid was washed with 1 L of water. The obtained solid was slurried in 10 L of water for 30 minutes, followed by filtration and washing the separated solid with 300 ml of methanol, then the solid was subjected to suction drying for about 1 hour. Wet compound was slurried in 5 L of methanol for 30 minutes. The obtained suspension was filtered and the solid was washed with 300 ml methanol. The wet solid was subjected to suction drying for 1 hour to afford 103 g of crude title compound. Purity: 92.24% by HPLC.

103 g of crude 7-ethyl 10-hydroxy camptothecin, prepared above, was suspended in 3 L of acetic acid and the temperature raised to 85° C. The resultant reaction solution was subjected to concentration at 50° C. under vacuum of 580 mm Hg until 500 ml of solution remained and then cooled to about 27° C. 1500 ml of methanol was added to the solution followed by stirring for about 30 minutes. The solid that separated was filtered and washed with 170 ml of methanol. The obtained solid was dried under a vacuum of 600 mm Hg for 1 hour. The above process was repeated one more time to afford 82 g of 7-ethyl 10-hydroxy camptothecin. Purity: 95.65% by HPLC.

82 g of 7-ethyl 10-hydroxy camptothecin, obtained above, was suspended in 2 L of N,N-dimethyl formamide (DMF) and the temperature raised to 80° C. with simultaneous stirring for 20 minutes. The resultant reaction solution was cooled to 0° C. and then stirred for 30 minutes. The separated solid was filtered and washed with 170 ml of methanol. The solid obtained was subjected to suction drying over a period of 1 hour. The obtained wet solid was slurried in 1 L of methanol for 30 minutes and then filtered and the solid was washed with 170 ml of methanol. The solid obtained was dried at 70° C. for 4 hours to afford 54.7 g of 7-ethyl 10-hydroxy camptothecin. Purity: 98.12% by HPLC.

### EXAMPLE 3

### PREPARATION OF 4-PIPERIDINOPIPERIDINE CARBAMOYL CHLORIDE

150 g of 4-piperidinopiperidine was dissolved in 10.5 L of dichloromethane and the solution was cooled to about 0° C. A solution of triphosgene (94.8 g of triphosgene in 1.2 L of dichloromethane) was added slowly to the solution at about 5° C. over a period of 45 minutes under a nitrogen atmosphere with simultaneous stirring. The resultant reaction mixture was stirred at 27° C. for 12 hours. The reaction mixture was filtered and then the filtrate was washed with 1.2 L of 7% sodium bicarbonate solution. The organic layer was separated and concentrated completely at about 40° C. under a vacuum of 580 mm Hg to afford title compound. Purity: 97.2% by GC.

### EXAMPLE 4

### Preparation of Irinotecan

4-piperidinopiperidine carbamoyl chloride obtained from Example 3 was dissolved in 3.24 L of pyridine, followed by the addition of 54 g (0.137 moles) of 7-ethyl 10-hydroxy camptothecin, prepared according to Example 2. The mixture was heated to 40° C. with simultaneous stirring for 2 hours under a nitrogen atmosphere. The reaction mixture was cooled to about 27° C.; then filtered through a celite bed and the celite was washed with 200 ml of pyridine. The filtrate was concentrated to get 250 ml solvent remaining at 50° C. under a vacuum of 650 mm Hg. The obtained residue was dissolved in 1 L of water and filtered through celite. The obtained filtrate was washed with 500 ml of n-heptane and with 2×500 ml of ethyl acetate. The aqueous layer was concentrated at 50° C. to a 250 ml volume remained and then codistilled with 2×1 L of isopropyl alcohol at 50° C. to get 500 ml solvent remaining. The obtained suspension was allowed to cool to room temperature and then filtered. The filtered solid was washed with 100 ml of isopropyl alcohol and finally subjected to drying at 70° C. for 3 hours under a vacuum of 650 mm Hg to afford 69.6 g of crude irinotecan. Purity: 94.7% by HPLC.

### EXAMPLE 5

### Preparation of Irinotecan

18 g of 4-piperidinopiperidine carbamoyl chloride, obtained according to Example 3, was dissolved in 637 ml of pyridine, followed by the addition of 10 g of 7-ethyl 10-hydroxy camptothecin, prepared according to Example 2. The mixture was heated to 40° C. with simultaneous stirring for 2 hours under a nitrogen atmosphere. The above reaction mixture was cooled to about 27° C., and then filtered through a celite bed. The filtrate was concentrated at 50° C. under a vacuum of 650 mm Hg to a volume of 40 ml. 185 ml of n-heptane was added to the residue and concentrated at 50° C. under vacuum of 580 mm Hg to a volume of 50 ml. The above step was repeated two more times and then 185 ml of water was charged to the resultant residue. Adjusted the pH of the reaction mixture to 5.7 with 1 ml of acetic acid and then separated the two layers, and washed the aqueous layer with 2×92.5 ml of ethyl acetate. The obtained aqueous layer was concentrated at 45° C. under a vacuum of 590 mm Hg to a volume of 50 ml. 185 ml of isopropyl alcohol was charged to the residue and concentrated to a volume of 50 ml. Again 185 ml of isopropyl alcohol was charged to the above obtained residue and concentrated at 45° C. under a vacuum of 580 mm Hg to a volume of 50 ml. The suspension was cooled to 27° C. and stirred for 1 hour. The obtained suspension was filtered and washed with 20 ml of isopropyl alcohol and the solid dried at 65° C. under a vacuum of 580 mm Hg for 4 hours to afford 11.5 g of the title compound. Purity: 96.47% by HPLC.

### EXAMPLE 6

### Purification of Irinotecan by Preparative Column Chromotography

150 g of crude irinotecan, prepared according to the procedure of Example 4, was introduced in 3 lots into a preparative column containing silica gel of particle size 500-750 mesh and eluted using methanol/dichloromethane as the eluent system.

The column was packed with 1.5 kg of silica gel slurried in methanol. Then the column was stabilized with isopropanol, and eluted with 50% methanol in dichloromethane for baseline checking for 40 minutes at a flow rate of 550 ml/minute. Then theoretical plates were calculated by injecting a pure irinotecan, which was prepared by dissolving 500 mg of pure irinotecan in 10 ml of 50% methanol in dichloromethane and the column was eluted for about 40 minutes with 50% methanol in dichloromethane. The column was considered to be suitable for purification if the theoretical plate count was more than 4000. Then 50 g of crude irinotecan was dissolved in 500 ml of 5% methanol in dichloromethane (25 ml of methanol and 475 ml of dichloromethane) and was injected into column and eluted with 100% dichloromethane for 0.00-4.00 minutes at a flow rate 300 ml/minute, 100% dichloromethane for 4.2-8.0 minutes at a flow rate of 550 ml/min, 5% methanol in dichloromethane for 8.2-50 minutes, 7% methanol in dichloromethane for 50.2-65 minutes, and finally eluted with 15% methanol in dichloromethane for 70.2-80 minutes.

While eluting the column, the fractions were collected as follows:

a) Impure fraction (I) was collected from start of the peak onward, around 3 minutes, until an increase in the absorption was observed;

b) Pure fraction (II) was collected after fraction (I) up to 28 minutes (total collection duration is 28 minutes);

c) Impure fraction (III) was collected after fraction (II) for another 3-5 minutes; and

d) Impure fraction (IV) was collected after completion of fraction (III) for another 10 minutes.

The above process was repeated with 2×50 g of irinotecan base to get pure fractions. The pure compound fractions were concentrated under a vacuum of 475±25 mm Hg at about 45° C. till to get 250 ml of solvent in solution remained and precipitated by adding 2.6 L of n-heptane to afford 87.25 g of irinotecan with a purity of 99.4% by HPLC.

### EXAMPLE 7

### Purification of Irinotecan by Preparative Column Chromotography

60 g of crude of irinotecan, prepared according to the procedure of Example 4, was subjected to preparative column chromatography by using 15-25 μm silica gel with methanol/dichloromethane as the eluent system.

The column was packed with 220 g of silica gel. Then the column was stabilized with 9 L of isopropanol, and eluted with 6 L of 10% dioxane in n-heptane for baseline checking. Then theoretical plates were calculated for column efficiency by injecting a mixture of dibutylphthalate, diethylphthalate, and dimethylphthalate (20 μl). Washed the column by running dichloromethane for 30 minutes with a flow rate of 150 ml/minute. 6×10 g of crude irinotecan was dissolved in 5% methanol/dichloromethane and injected into the column followed by elution with dichloromethane for 10 minutes followed by 5% methanol/dichloromethane as elutant to collect 3.915 L of desired pure fraction after 3 minutes of main peak started. The organic solution was concentrated under a vacuum of about 500 mm Hg at 48° C. until the solution volume was 30 ml and a solid was precipitated by adding 130 ml of n-heptane. The obtained suspension was stirred for 30 minutes and filtered. The solid was washed with 33 ml of n-heptane and dried at 45° C. under a vacuum of 500 mm Hg for 1 hour to afford 36.8 g of irinotecan with a purity by HPLC of 99.64%.

### EXAMPLE 8

### Purification of Irinotecan Using Conventional Column Chromatography

Prepared a silica gel slurry by mixing of 48 L of dichloromethane and 24 Kg of 100-200 mesh silica gel in a clean container. Charged the silica gel slurry into the column and conditioned the column by recycling the dichloromethane to get a flow rate of 60 L/hour through the column. 0.9 Kg of irinotecan was dissolved in 20 L of a 3% methanol in dichloromethane mixture that was prepared by mixing 0.6 L of methanol and 19.4 L of dichloromethane in a flask. Concentrated the solution to 4.5 L at 47° C. under vacuum of 570 mm Hg and then charged the concentrated mixture to the silica gel bed in the column. Rinsed the flask with 0.25 L of dichloromethane and charged the rinse solution to the silica gel bed, and then charged 1 Kg of silica gel into the column. Placed a thick layer of cotton over the silica gel and then eluted the column with 60 L of dichloromethane. Eluted the column with mixtures of methanol in dichloromethane: 3% for 50 L; 5% for 150 L; 6% for 150 L; 7% for 250 L; 8% for 50 L; 10% for 30 L; and 15% for 50 L. Collected pure fractions from elution with 6%, 7%, and 8% of methanol in dichloromethane and then these fractions were concentrated to a 4.5 L volume remained. Solid was precipitated from the concentrated solution by adding 18 L of n-heptane and the obtained suspension was filtered. The obtained solid was dried under a vacuum of 550 mm Hg for 1 hour at 45° C. to afford 522 g of title compound.

### EXAMPLE 9

### Purification of Irinotecan Using Ethanol

36.8 g of irinotecan prepared according to the procedure of Example 7 was dissolved in 184 ml of 5% methanol in dichloromethane with stirring for about 10 minutes to afford a clear solution. 368 ml of ethanol was added to the above solution and concentrated at 50° C. to a volume of 184 ml remaining. 184 ml of ethanol was added to the solution and concentrated at 50° C. to get 184 ml of solution remaining. The above step was repeated and then the solution allowed to cool to room temperature with simultaneous stirring over a period of 60 minutes. The mixture was filtered and the solid washed with ethanol, and then the solid was dried to a moisture content below 5% as determined by the Karl Fischer (“KF”) method to afford 30.2 g of irinotecan with a purity of 99.9% by HPLC.

### EXAMPLE 10

### Preparation of Irinotecan Hydrochloride Trihydrate

25 g of irinotecan prepared according to the procedure of Example 9 was slurried with 250 ml of water for about 30 minutes and filtered, the solid was washed with 12.5 ml of water, 27.5 ml of 5N hydrochloric acid was added to the filtrate, and the filtrate was heated to 70° C. with simultaneous stirring for 3 hours at 70° C. until complete precipitation occurred. The reaction mass was cooled to 30° C. and filtered, followed by washing the solid with 7 ml of water and 15 ml ethanol. Finally the solid was dried under a vacuum of about 650 mm Hg at 30° C. for 3 hours to afford 22.5 g of the title compound with a purity by HPLC of 99.95%.

### EXAMPLE 11

### Preparation of Irinotecan Hydrochloride Trihydrate

290 g of irinotecan prepared according to the procedure of Example 9 was slurried with 2.9 L of water and 87 ml of 5N hydrochloric acid was added over about 30 minutes. The mixture was filtered through a 0.22 micron filter, and then 232 ml of 5N hydrochloric acid was added to the filtrate. The filtrate was heated to 70° C. with simultaneous stirring and stirred for about 3 hours at 70° C. until precipitation occurred. The reaction mass was cooled to 30° C. and filtered, followed by washing the solid with 87 ml of water and 0.174 L of ethanol. Finally the solid was dried under a vacuum of not less than 580 mm Hg at 30° C. for about 3 hours to afford 257 g of the title compound.

Purity by HPLC: 99.81%; impurities by HPLC (“LOD” is the lower limit of detection and “LOQ” is the lower limit for quantification):

| Impurity Name | Result | LOD (%) | LOQ (%) |
|---|---|---|---|
| Camptothecin | Not detected | 0.0015 | 0.005 |
| 10-Hydroxy camptothecin | 0.01% | 0.0015 | 0.005 |
| 7-Ethyl-10-hydroxy camptothecin | 0.03% | 0.0015 | 0.005 |
| 7-Ethyl camptothecin | Not detected | 0.005 | 0.015 |
| 7,11-Diethyl-10-hydroxy | Not detected | 0.0025 | 0.0075 |
| camptothecin | | | |
| 10-[4-(1-piperidino)-piperidino]- | Not detected | 0.003 | 0.01 |
| carbonyloxy camptothecin | | | |
| 7,11-Diethyl-10-[4(1-piperidino)- | 0.05% | 0.005 | 0.015 |
| 1-piperidino]- | | | |
| carbonyloxycamptothecin | | | |

Water content by KF (Karl Fischer method): 8.1% w/w.

The XRPD diffraction pattern was in accordance with FIG. 1.

Residual solvents content were determined by gas chromatography with samples dissolved in dimethylsulfoxide.

| Solvent | Content (ppm) |
|---|---|
| Methanol | 210 |
| Ethanol | 251 |
| Isopropyl alcohol | 13 |
| Dichloromethane | 30 |
| Ethyl acetate | 1.6 |
| n-Heptane | 0.04 |
| Pyridine | 0.5 |
| DMF | 31 |

We claim:

1. A process for preparing irinotecan or a salt thereof, comprising purifying 7-ethyl-10-hydroxycamptothecin by:
i) slurrying 7-ethyl-10-hydroxycamptothecin in an alcohol; then
ii) dissolving 7-ethyl-10-hydroxycamptothecin in acetic acid, removing acetic acid to form a concentrated solution, and combining with an alcohol to form a precipitate; then
iii) recrystallizing 7-ethyl-10-hydroxycamptothecin.

2. The process of claim 1, wherein an alcohol for i) is a C₁-C₆ alkyl alcohol.

3. The process of claim 1, wherein an alcohol for ii) is a C₁-C₆ alkyl alcohol.

4. The process of claim 1, wherein an alcohol for i) comprises methanol.

5. The process of claim 1, wherein an alcohol for i) comprises methanol.

6. The process of claim 1, wherein recrystallization in iii) is from a solution comprising N,N-dimethylformamide, dimethylsulfoxide, or N,N-dimethylacetamide.

7. The process of claim 1, wherein recrystallization in iii) is from a solution comprising N,N-dimethylformamide.

8. The process of claim 1, further comprising reacting crystallized 7-ethyl-10-hydroxycamptothecin with 4-piperidinopiperidinecarbamoyl chloride to form irinotecan.

9. A process for purifying irinotecan, comprising adding a solution comprising irinotecan to a column of silica gel, eluting using dichloromethane, then eluting using an eluent comprising a mixture of methanol and dichloromethane.

10. The process of claim 6, wherein a mixture of methanol and dichloromethane comprises about 5 percent by volume methanol.

11. A process for the preparation of irinotecan hydrochloride trihydrate comprising reacting irinotecan with hydrochloric acid in water.

12. Irinotecan hydrochloride trihydrate prepared by the process of claim 1 and containing:
less than about 0.15% by weight of (S)-4-ethyl-4-hydroxy-1H-pyrano[3′, 4′:6,7]indolizino(1,2-b)quinoline-3,14(4H,12H)-dione;
less than about 0.15% by weight of (S)-4-ethyl-4,9-dihydroxy-1H-pyrano[3′,4′:6,7]indolizino(1,2-b)quinoline-3,14(4H,12H)-dione;
less than about 0.15% by weight of (S)-4,11-diethyl-4,9-dihydroxy-1H-pyrano[3′,4′:6,7]indolizino(1,2-b)quinoline-3,14(4H,12H)-dione;
less than about 0.15% by weight of (S)-4,11-diethyl-4-hydroxy-1H-pyrano[3′,4′:6,7]indolizino(1,2-b)quinoline-3,14(4H,12H)-dione;
less than about 0.15% by weight of (S)-4,8,11-triethyl-4,9-dihydroxy-1H-pyrano[3′,4′:6,7]indolizino(1,2-b)quinoline-3,14(4H,12H)-dione;
less than about 0.15% by weight of (S)-4-ethyl-4,9-dihydroxy-1H-pyrano[3′,4′:6,7]indolizino(1,2-b)quinoline-3,14(4H,12H)-dione-9-(1,4-bipiperidine)-1′-carboxylate; and
less than about 0.15% by weight of (S)-4,8,11-triethyl-4,9-dihydroxy-1H-pyrano[3′,4′:6,7]indolizino(1,2-b)quinoline-3,14(4H,12H)-dione-9-(1,4′-bipiperidine)-1′-carboxylate.

13. Irinotecan hydrochloride trihydrate of claim 9, containing less than about 0.005% by weight of (S)-4,8,11-triethyl-4,9-dihydroxy-1H-pyrano[3′,4′:6,7]indolizino(1,2-b)quinoline-3,14(4H,12H)-dione-9-(1,4′-bipiperidine)-1′-carboxylate.
